(19) United States
(12) Patent Application Publication
(10) Pub. No.: US 2002/0087093 A1
(43) Pub. Date: Jul. 4, 2002

(54) Body fat analyzer with wireless remote

(76) Inventor: Sunny Chai, Hong Kong (HK)

Correspondence Address: PENNIE AND EDMONDS, 1155 AVENUE OF THE AMERICAS, NEW YORK, NY, 100362711

(21) Appl. No.: 10/081,789

(22) Filed: Feb. 22, 2002

Related U.S. Application Data
(60) Provisional application No. 60/284,228, filed on Apr. 16, 2001.
(60) Provisional application No. 60/284,541, filed on Apr. 17, 2001.

(51) Int. Cl. A61B 5/05

(52) U.S. Cl. 600/547; 128/903

(57) ABSTRACT

A bioelectrical-impedance-based body fat analyzer is provided that has a base unit for measuring weight and body impedance and a remote unit for inputting data and determining the body fat percent of a patient. The two units are connected via a wireless communications link formed by wireless ports on the base unit and the remote unit. There is further provided a method of operating a body fat analyzer having both base and remote units, comprising the steps of (a) measuring, at a base unit, an attribute of the patient's body; (b) inputting, at a remote unit, patient data such as gender, height, and body type; (c) calculating, based upon both the data and the measured attribute, a value representing the body composition of such patient; and (d) outputting, at the remote unit, the calculated value.

This application claims the benefit under 35 U.S.C. §119 of U.S. Provisional Application No. 60/284,228 filed Apr. 16, 2001 and U.S. Provisional Application No. 60/284,541 filed Apr. 17, 2001.

### FIELD OF THE INVENTION

The present invention relates to a body fat analyzer and, more particularly, to a bioelectrical-impedance-based body fat analyzer having a base unit for measuring weight and body impedance and a remote unit for calculating and outputting the body fat percent.

### BACKGROUND OF THE INVENTION

Body composition, and in particular percent body fat, is a well-recognized measure of physical health. A known technique to measure percent body fat is bioelectric impedance measurement, as described, for example, in U.S. Pat. No. 5,611,351 to Sato et al. In this technique, a person whose body fat is to be measured (“the subject”) stands upon a device that has four electrodes mounted on its upper surface. Then, a constant current source produces a 50 kHz, 800 microampere electric current that passes first through two electrodes in contact with the subject's toes and then through two or more reference resistors that are located in series with the subject's body and with each other. The electric current flowing through the subject causes a voltage potential to develop across the subject's heels.

Next, the heel-to-heel voltage is measured via two other electrodes in contact with the subject's heels, and the voltages across the reference resistors are also measured while the electric current is applied to the subject's toes. A microprocessor then compares the voltages measured across the reference resistors with the heel-to-heel voltage to derive the heel-to-heel impedance. After certain additional parameters such as age, weight, and height are entered via a keyboard connected to the microprocessor, it calculates body density using an algorithm relating body impedance and the additional parameters to body density. Once body density is obtained, the microprocessor performs a second calculation to convert body density to percent body fat and outputs the result via an LCD display.

It is also known to mount the four electrodes in a base unit upon which the subject stands, to provide a remote handheld unit that contains the other components, and to connect the two units by a cable, as shown in FIG. 1. See, for example, U.S. Pat. No. 6,088,615 to Masuo. The present inventor has found, however, that using a cable to connect a remote unit to a base unit can cause the following problems:

First, the cable introduces measurement errors in three ways: (a) it contributes to the impedance measured by the handheld unit; (b) cross-talk may occur between the current-carrying conductors and the voltage-measuring conductors in the cable; and (c) the cable may act as an antenna and receive electromagnetic noise interference (EMI) signals. EMI noise signals can be generated, e.g., by power cabling, transformers, large motors, and radio transmitters. Such signals can be a very serious problem, because the voltages being measured by the body fat analyzer are quite small (i.e., in approximately the range from 0.1-10 millivolts) and the amplitude of the noise can be quite large by comparison.

Second, the cable decreases the reliability of the system by introducing a point of possible failure. The cable may be broken by the repeated stresses that occur as the body fat analyzer is used. Moreover, the cable can become separated from the base and handheld units and be lost.

Third, a design compromise must be made in selecting the length of the cable. If the cable is long, it is bulky, hard to manage and store, and relatively heavy. But if the cable is short, the distance at which the handheld unit can be operated is correspondingly limited.

This limited distance between the base unit and the remote unit may create a problem in situations in which physical separation of the base unit and the remote unit is needed. In prisons or hospitals, for example, it may be preferable to limit the physical interaction of dangerous inmates or patients with medical staff, by locating the base unit in one room while operating the remote unit from another room. Under such circumstances, a prudent building manager or designer (and perhaps local electrical codes) may require that a hard-wired connection be provided between the two units via permanently-installed telecommunications terminal boxes and telecommunications cabling. If so, the cost of installing the body fat analyzer can be quite high.

Similarly, in some applications—for example, in an educational setting—it may be desirable to locate the base unit some distance away from the remote unit, so that the patients (students) can access the base unit in one part of a room while an administrator records the information in another part. In this way, the body composition measurements of each patient could be more easily kept confidential.

### SUMMARY OF THE INVENTION

The present invention avoids or minimizes the above-described problems. The objective of the invention is to provide a communications link between a base unit and a remote unit of a body fat analyzer that (1) does not require a physical connection between the two units, and (2) does not introduce error into the body fat measurements.

These and other features are provided in accordance with the method and apparatus of the invention. In a preferred embodiment of the invention, an apparatus for measuring the body composition of a patient in accordance with the invention comprises: (a) a base unit having two or more electrodes, and a measurement circuit that measures via the electrodes at least one body attribute (e.g., body impedance) of the patient; (b) a remote unit having a display and at least one data input port (such as a serial port or a keypad) at which data concerning such patient may be input; (c) a wireless communication link connecting said base unit and said remote unit; and (d) a microprocessor located within one of said base unit and said remote unit, wherein the microprocessor receives at least one datum from said data input port, calculates, based upon both the datum and the body attribute measured by said measurement circuit, a value representing the body composition of such patient, and outputs the calculated value. In a preferred embodiment, the measuring circuit comprises a current source that supplies an electrical current through at least two of said two or more electrodes and a voltage detector that is configured to detect a voltage across at least two of said two or more electrodes.

There is additionally disclosed a method for measuring the body composition of a patient in accordance with the invention, comprising the steps of: (a) measuring, at the base unit, an attribute (such as body impedance) of the patient's body; (b) inputting, at a remote unit, at least one datum concerning such patient; (c) calculating, based upon both the datum and the measured attribute, a value representing the body composition of such patient; and (d) outputting, at the remote unit, the calculated value.

### BRIEF DESCRIPTION OF THE DRAWINGS

FIG. 1 is a diagram of a prior art body fat analyzer.

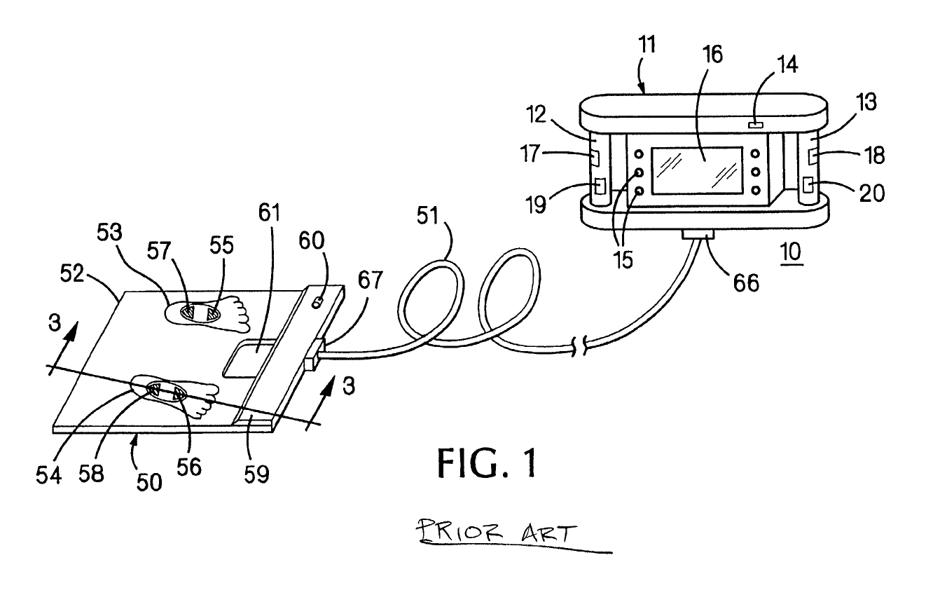

FIG. 2 is a rendering of a preferred embodiment of a body fat analyzer according to the present invention.

FIG. 3 is a schematic block diagram of an embodiment of a body fat analyzer according to the present invention.

FIG. 4 is a flow chart of the operation of the microprocessor in the base unit of the embodiment shown in FIG. 3.

FIG. 5 is a flow chart of the operation of the microprocessor in the remote unit of the embodiment shown in FIG. 3.

FIG. 6 is a schematic block diagram of an alternative embodiment of the present invention.

### DETAILED DESCRIPTION OF THE INVENTION

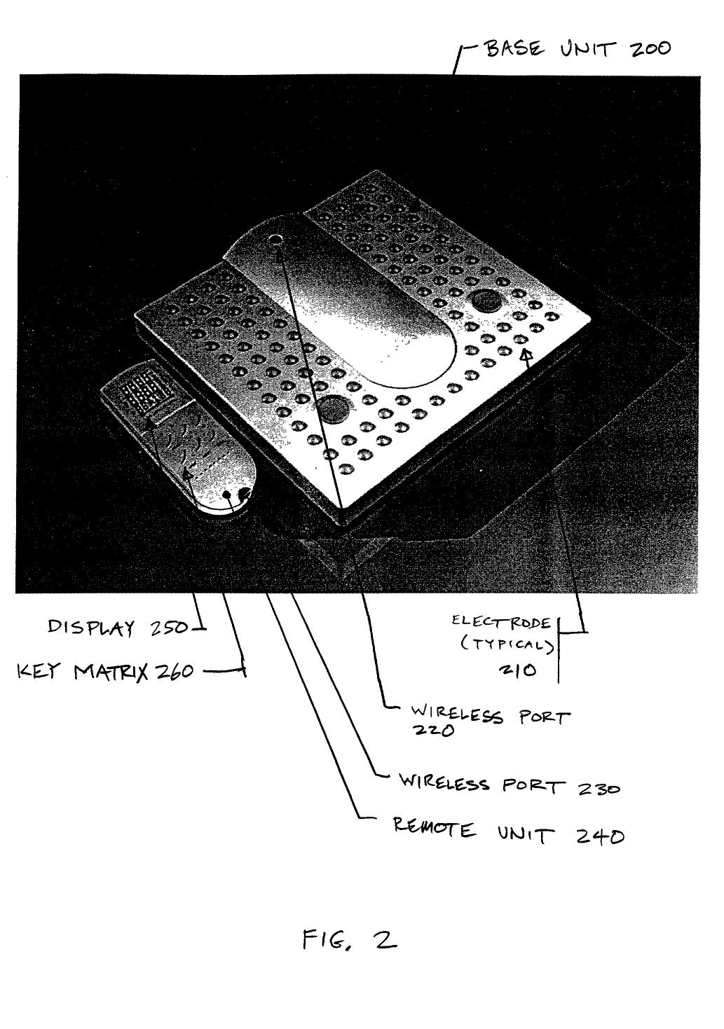

A device embodying the present invention, as shown in a preferred embodiment in FIG. 2, comprises a base unit 200 and a remote unit 240, connected via a wireless communications link formed by a wireless port 220 on the base unit and a wireless port 230 on the remote unit. Base unit 200 preferably includes at least two electrodes 210 that are used to measure an attribute of the patient, such as, i.e., the patient's body impedance, which may be used to calculate the patient's body composition. Remote unit 240 comprises a key matrix 260 and an LCD display 250.

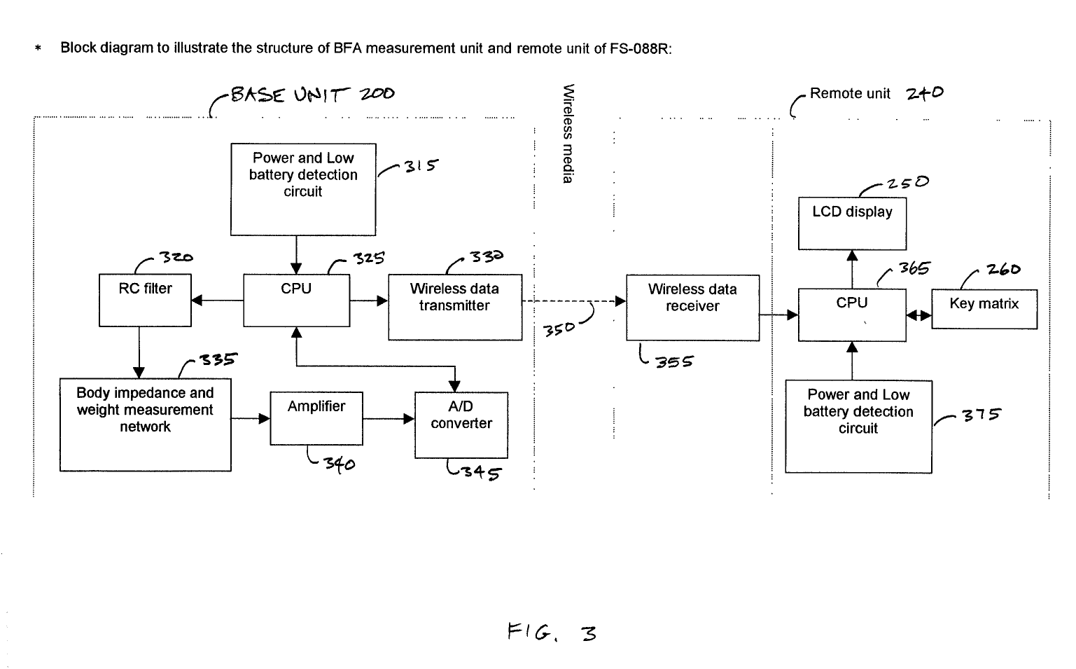

With reference to FIG. 3, which is a block diagram of the system shown in FIG. 2, base unit 200 further comprises a microprocessor-based CPU 325 that controls the operation of base unit 200. Regulated power is supplied via power and low battery detection circuit 315. Base unit 200 also includes a measurement circuit configured to measure the attribute to be used in calculating the body composition.

In a preferred embodiment, the measurement circuit is a circuit configured to measure body impedance. Preferably, the circuit includes a stable current source (not shown) to supply an AC current (preferably having a frequency of 50 kHz) to a body impedance and weight measurement network 335. If the stable current source is capable of producing only a square wave signal, an RC filter 320 may be additionally provided to convert the square wave AC current signal to a sinusoidal current signal. The current signal, when passed through the electrodes (210 in FIG. 2) in contact with the patient's feet, causes an AC voltage to develop across the feet that is proportional to the impedance of the patient's body.

In addition, in a preferred embodiment, base unit 200 also includes a weight measurement platform (not shown) that contacts a weight sensor (not shown), such as a strain gauge or force sensitive resistor. If so, the measurement circuit includes circuitry to produce a voltage across the sensor that is proportional to the weight of the patient. Such circuitry and sensors are presently in use in electronic weight scales and well-known to those of skill in the art.

The measurement circuit further may comprise an amplifier 340 to amplify the measured attributes and an A/D converter to convert them to digital form. In a preferred embodiment, base unit CPU 325 then calculates the body weight and the body impedance from the measured voltage values via techniques that are well-known in the art.

In accordance with the invention, the measured attributes (weight and impedance) are transmitted to remote unit 240 via a wireless data transmitter 330 and wireless media 350. In a preferred embodiment, transmitter 330 is a radio-frequency (“RF”) transmitter. This is a preferred embodiment, since RF transmissions are omnidirectional and not easily blocked by, i.e., internal walls in a building. In another embodiment, transmitter 330 is an ultrasonic transmitter (which is also omnidirectional but more limited in range than an RF transmitter because it is blocked by a building's internal walls). In yet another embodiment, the transmitter is an IR transmitter.

The wireless transmission scheme may be either analog or digital. For example, in one embodiment of the invention, the measured voltages are transmitted in digital form over a 33 kHz carrier signal that is modulated at a rate of 1 kHz using amplitude-shift keying. Other transmission schemes known to those of ordinary skill in the art, such as frequency or amplitude modulation, may also be employed.

The wireless transmission is received at remote unit 240 by a wireless receiver that corresponds in type (RF, IR, or ultrasound) to transmitter 330, and the signal representing the measured attribute(s) is passed in turn to the remote unit CPU 365. Based on the measured attribute(s) and on patient information (such as height, age, sex, and body type) that may be entered via key matrix 260, remote unit CPU 365 calculates the body fat percent and outputs the calculated value on LCD display 250.

Those of skill in the art will recognize that, although the wireless communication link 350 is shown as unidirectional, a bidirectional link may also be employed. If so, it is then possible to transmit data entered at key matrix 260 in remote unit 240 to base unit CPU 325, which could then perform the calculation of body fat instead of remote unit CPU 365. In this alternative embodiment, the calculated value would then be transmitted from base unit 200 to remote unit 240 for display on LCD display 250.

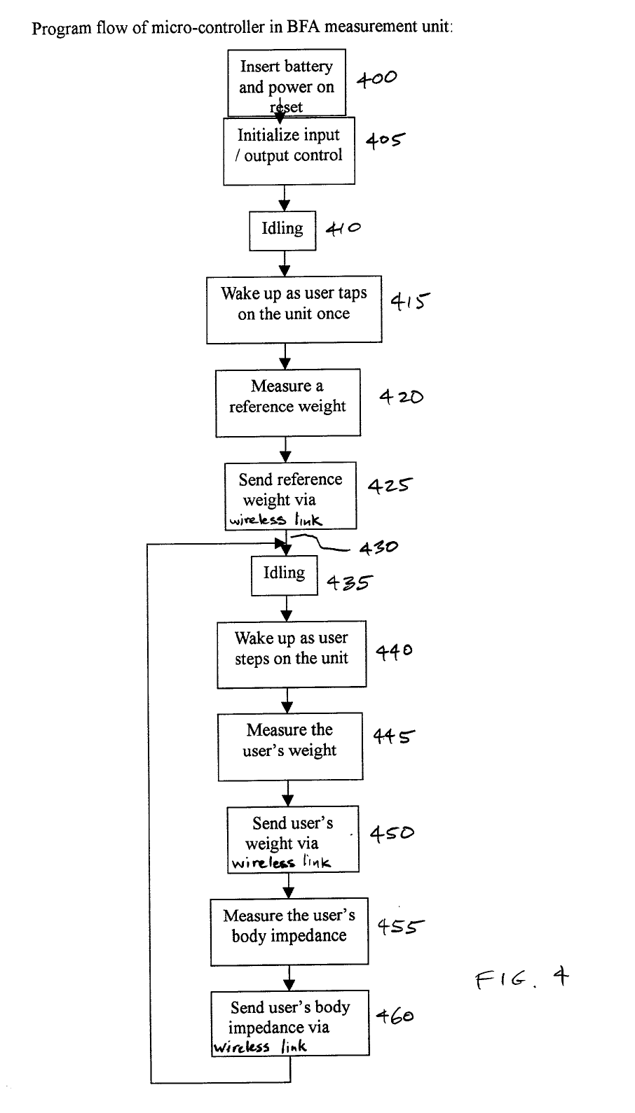

A flowchart showing the operation of base unit CPU 325 in more detail is shown in FIG. 4. At step 400, a battery is inserted into base unit 200. Power is supplied to base unit CPU 325, and a watchdog circuit sends a reset signal to base unit CPU 325. Base unit CPU 325 accordingly resets, initializes its input/output control circuitry at step 405, and then idles at step 410. After a patient taps on the top of the base unit (which action is detected by base unit CPU 325 as a momentary change in the weight measured by the weight measuring circuitry), base unit CPU 325 measures a reference (tare) weight at step 420 and transmits it to remote unit 240 via the wireless link (step 425). At step 435, base unit CPU returns to an idle state. Next, after a patient steps on base unit 200, base unit CPU wakes up (step 440) and measures his weight (step 445). At step 450, it sends the measured weight to remote unit 240 and proceeds at step 455 to measure the patient's body impedance, which it also transmits to remote unit 240 (step 460). Finally, it returns via step 430 to an idle state (step 435).

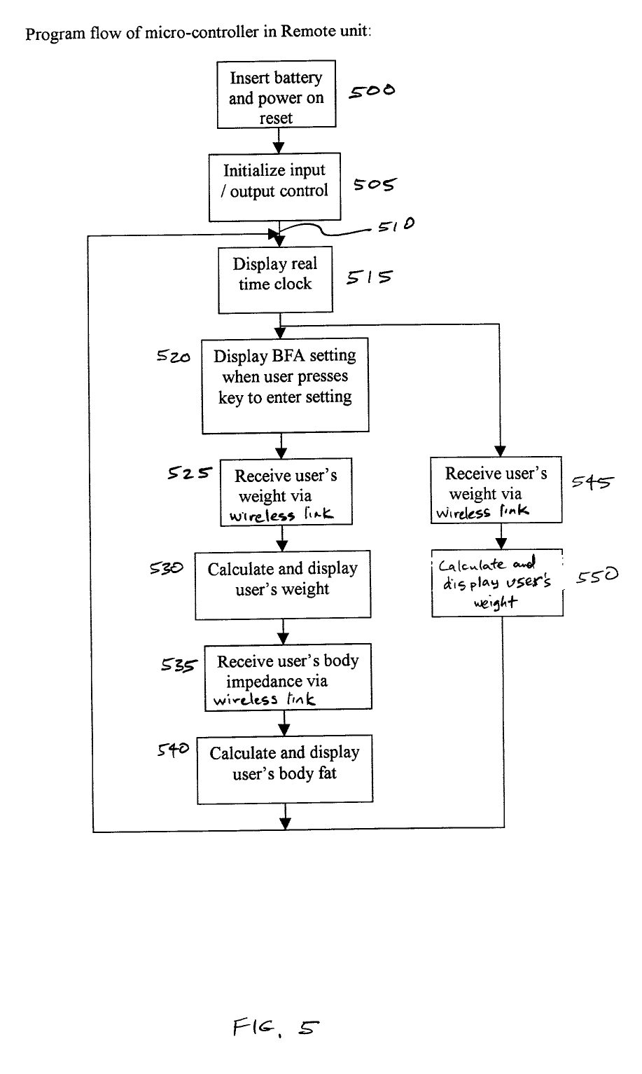

The operation of remote unit CPU 365 is shown in FIG. 5. At step 500, a battery is inserted into remote unit 240, supply power is applied to remote unit CPU 365 and another watchdog circuit sends a reset signal to remote unit CPU 365. At step 505, remote unit CPU 365 initializes its input/output control circuitry. It then displays the time of day in step 515 until one of the following two events occur:

(1) If a patient presses a certain key, remote unit CPU 365 enters a “data entry” mode and prompts the patient to enter the data (age, gender, height, etc.) needed to calculate his body fat (step 420). Alternatively, another key may be pressed that causes remote unit CPU 365 to recall such data from the CPU's memory circuits. When the patient then stands on base unit 200, it measures his weight in kilograms and transmits it to remote unit 240. At step 525, remote unit CPU 365 receives the patient's weight (in kilograms), and, at step 530, converts the weight to a patient-selected unit of weight (pounds, etc.) and outputs it on LCD display 250. At step 535, it receives the patient's body impedance (transmitted by base unit 200 at step 460 above) and proceeds at step 540 to calculate and output the patent's percent body fat on LCD display 250.

(2) If remote unit CPU 365 receives a weight measurement (step 545) (in kilogram) transmitted to it by base unit 200, it converts the weight in kilograms to the patient-selected unit of weight and displays the weight at step 550 and, returning to flow point 510, continues to display the clock at step 515.

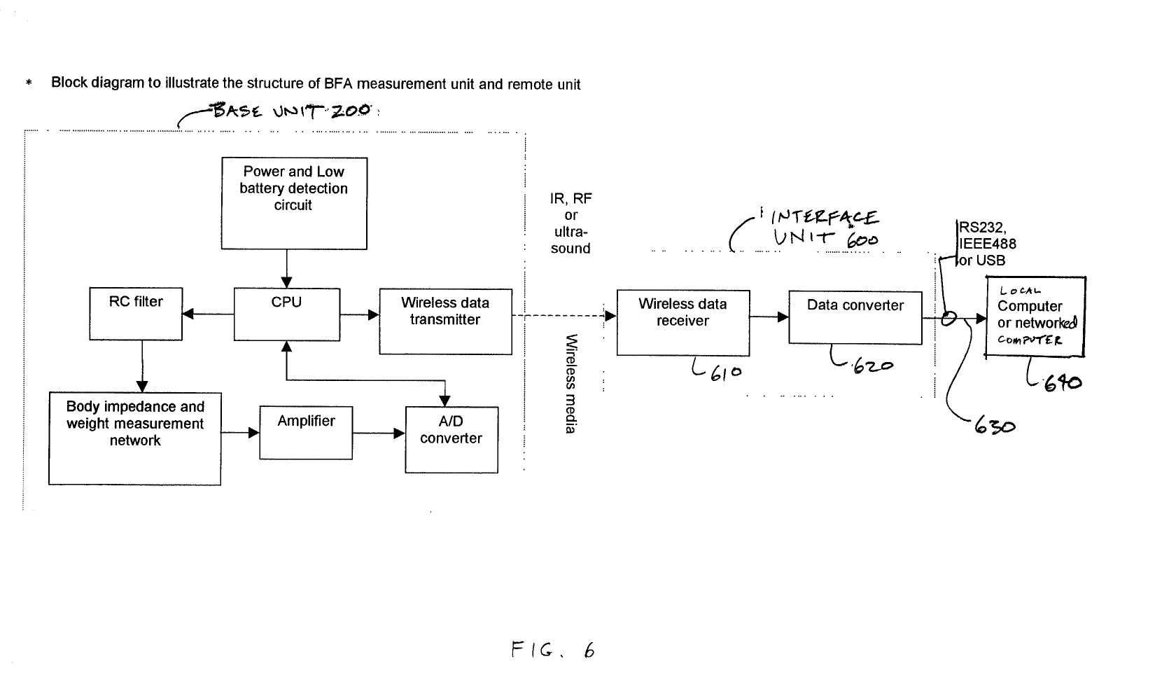

A second preferred embodiment of the present invention is shown in FIG. 6. In this embodiment, the remote unit CPU 365, LCD display 250, and key matrix 260 of FIGS. 2 and 3 are integrated into a computer 640, which is connected to base unit 200 via a wireless interface unit 600. This interface unit comprises a wireless data receiver 610 and a data converter circuit 620 of the type customarily used to interface a digital signal to, i.e., an RS-232, IEEE 488, or USB port on a local personal computer or a network server. The flow of operation in this second preferred embodiment is similar to that described above with reference to FIGS. 2-5, except that in this embodiment the computer performs the steps previously carried out by remote unit CPU 365. Preferably, computer 640 is capable of storing a patient's input data (i.e., gender, height, and body type) and measured weight and percent body fat in a database of patients and creating individual or group reports that may be printed on a printer (not shown).

While the invention has been described with reference to a specific embodiment, it will be appreciated by those of ordinary skill in the art that modifications can be made to the structure and form of the invention without departing from its spirit and scope, which is defined in the following claims.

What is claimed is:

1. An apparatus for measuring the body composition of a patient, comprising:
(a) a base unit, having
(1) two or more electrodes, and
(2) a measurement circuit, configured to measure at least one attribute of the patient via said two or more electrodes;
(b) a remote unit, having
(1) a display, and
(2) at least one data input port configured to receive data concerning such patient;
(c) a wireless communication link connecting said base unit and said remote unit; and
(d) a microprocessor located within one of said base unit and said remote unit, wherein said microprocessor is configured:
(1) to receive at least one datum from said data input port,
(2) to calculate, based upon both an inputted datum and the measured attribute, a value representing the body composition of such patient, and
(3) to output the calculated value.

2. The apparatus of claim 1, wherein said measurement circuit comprises:
(a) a current source; and
(b) a voltage detector;
and wherein said current source supplies electrical current through at least two of said two or more electrodes and causes a voltage to develop across at least two of said two or more electrodes, and said voltage detector detects the voltage.

3. The apparatus of claim 1, wherein said data input port is one of a keypad and a communication port.

4. The apparatus of claim 1, wherein said wireless communication link comprises:
(a) a wireless transmitter located at said base unit; and
(b) a wireless receiver located at said remote unit.

5. The apparatus of claim 4, wherein said transmitter produces one of radio-frequency signals, infrared signals, and ultrasonic signals.

6. The apparatus of claim 1, wherein the remote unit is handheld.

7. The apparatus of claim 1, wherein the remote unit is one of a personal computer and a network server.

8. The apparatus of claim 7, wherein said wireless communication link comprises:
(a) a wireless transmitter located at said base unit; and
(b) a wireless interface unit in communication with said remote unit and having a wireless receiver configured to receive signals from said wireless transmitter.

9. The apparatus of claim 8, wherein said wireless interface unit further comprises a data converter.

10. The apparatus of claim 9, wherein said data converter converts data that is received by said wireless receiver to a format that may be input to said remote unit.

11. The apparatus of claim 9, wherein said data converter comprises one of an RS-232 port, an IEEE 488 port, or a USB port.

12. A method for determining the body composition of a patient, comprising the steps of:
(a) measuring, at a base unit, an attribute of the patient's body;
(b) inputting, at a remote unit, at least one datum concerning such patient;
(c) calculating, based upon both an inputted datum and the measured attribute, a value representing the body composition of such patient; and
(d) outputting, at the remote unit, the calculated value.

13. The method of claim 12, wherein said measuring step comprises the steps of:
(a) supplying, at a base unit, an electrical current through such patient's body; and
(b) detecting, at the base unit, a voltage between at least two points on such patient's body.

14. The method of claim 12, further comprising the step of communicating one of the inputted datum and the measured attribute from one unit to the other.

15. The method of claim 14, wherein said step of communicating comprises the steps of transmitting a wireless signal and receiving the wireless signal.

16. The method of claim 15, wherein said wireless signal is transmitted at one of a radio frequency, an infrared frequency, and an ultrasonic frequency.

17. The method of claim 15, further comprising the step of converting at least some of the information contained in the wireless signal to a format that may be input to a computer.

18. The method of claim 17, wherein the format is one of an RS-232 format, an IEEE 488 format, and a USB format.
